(19) United States
(12) Patent Application Publication
(10) Pub. No.: US 2025/0135207 A1
(43) Pub. Date: May 1, 2025

(54) SENSING CONFIGURATIONS FOR NON-UNIFORM NEUROSTIMULATION PATTERNS

(71) Applicant: Boston Scientific Neuromodulation Corporation, Valencia, CA (US)

(72) Inventors: Andrew James Haddock, Los Angeles, CA (US); Jessica Block, Los Angeles, CA (US)

(21) Appl. No.: 18/915,089

(22) Filed: Oct. 14, 2024

Related U.S. Application Data
(60) Provisional application No. 63/545,684, filed on Oct. 25, 2023.

(51) Int. Cl. A61N 1/36; A61N 1/372

(52) U.S. Cl. CPC A61N 1/36139; A61N 1/36175; A61N 1/37247

(57) ABSTRACT

A system may control when, with respect to a non-uniform waveform pattern used to deliver neurostimulation, to sense neural signals to appropriately control neurostimulation. The system may include a neural sensor configured to sense a neural signal, a neurostimulator configured to access a non-uniform waveform pattern and deliver the neurostimulation corresponding to the non-uniform waveform pattern, and a controller. The controller may be configured to automatically assign, based on the non-uniform waveform pattern, at least one sensing window for the non-uniform waveform pattern, control the neural sensor to sense the neural signal during the sensing window when the neurostimulation is delivered, and control the delivery of the neurostimulation from the neurostimulator based on the sensed neural signal.

### TECHNICAL FIELD

This document relates generally to medical systems, and more particularly, but not by way of limitation, to systems, devices, and methods for determining sensing configurations for neurostimulation delivered using non-uniform waveform patterns.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). For example, an implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device may be used to program the implantable neurostimulator with stimulation parameters controlling the delivery of the neurostimulation energy.

For example, the neurostimulation energy may be delivered in the form of electrical neurostimulation pulses and controlled using stimulation parameters that specify spatial (where to stimulate), temporal (when to stimulate), and informational (patterns of pulses directing the nervous system to respond as desired) aspects of a pattern of neurostimulation pulses. The neurostimulation signal may be referred to as a waveform, and a collection of pulses may be referred to as a waveform pattern. Many current neurostimulation systems are programmed to deliver periodic pulses with one or a few uniform waveform patterns continuously or in bursts. However, the human nervous systems use neural signals having much more sophisticated patterns to communicate various types of information, including sensations of pain, pressure, temperature, and the like. The nervous system may interpret an artificial stimulation with a simple pattern of stimuli as an unnatural phenomenon and respond with an unintended and undesirable sensation and/or movement.

Systems have been developed to use non-uniform waveform patterns to deliver neurostimulation. For example, the efficacy and efficiency of certain neurostimulation therapies may be improved and the side-effects may be reduced using non-uniform waveform patterns, which may more closely emulate natural neural signal patterns. Such systems may allow a user to customize the waveform pattern.

Sensing may be used to augment and adapt non-uniform waveform patterns much in the same way it can for uniform waveform patterns (also referred to as tonic stimulation programs). More particularly, sensed neural signals may quickly respond to the delivered neurostimulation. Tonic stimulation programs have a consistent delivery of neurostimulation energy. However, neurostimulation delivered using non-uniform waveform patterns do not have a consistent delivery of neurostimulation energy. Therefore, unlike for tonic stimulation programs, it may be important to know when to sense to control neurostimulation that is being delivered using non-uniform waveform patterns (i.e., which pulses in the pattern are to be used for “sense events”).

### SUMMARY

An example (e.g., Example 1) of a system includes a neural sensor configured to sense a neural signal, a neurostimulator configured to access a non-uniform waveform pattern and deliver the neurostimulation corresponding to the non-uniform waveform pattern, and a controller. The controller may be configured to automatically assign, based on the non-uniform waveform pattern, at least one sensing window for the non-uniform waveform pattern, control the neural sensor to sense the neural signal during the sensing window when the neurostimulation is delivered, and control the delivery of the neurostimulation from the neurostimulator based on the sensed neural signal.

In Example 2, the subject matter of Example 1 may optionally be configured such that the neural sensor is configured to sense evoked compound action potentials (ECAPs) or local field potentials.

In Example 3, the subject matter of any one or more of Examples 1-2 may optionally be configured such that the non-uniform waveform pattern includes activation pulses that cause a neural response and sub-activation threshold pulses that do not cause the neural response, and the controller is configured to automatically assign the sensing window by determining at least one pulse in the non-uniform waveform pattern that corresponds to a neural activation and assigning the at least one sensing window to sense the neural response to the neural activation.

In Example 4, the subject matter of Example 3 may optionally be configured such that the controller is configured to determine the at least one pulse in the non-uniform waveform pattern using a relationship between a neural activation and at least one of a pulse amplitude, a pulse frequency or a pulse width for the at least one pulse.

In Example 5, the subject matter of any one or more of Examples 3-4 may optionally be configured such that the controller is configured to determine the at least one pulse by automatically determining a largest pulse amplitude in the non-uniform waveform pattern, and the sensing window is automatically assigned to sense a neural response to the neurostimulation corresponding to the largest pulse amplitude in the non-uniform waveform pattern.

In Example 6, the subject matter of any one or more of Examples 3-4 may optionally be configured such that the controller is configured to automatically select multiple pulses in the non-uniform waveform pattern, and the sensing window is assigned to sense a neural response to the neurostimulation corresponding to the automatically selected multiple pulses.

In Example 7, the subject matter of Example 6 may optionally be configured such that the non-uniform waveform pattern is repeated to provide multiple instances of each pulse in the non-uniform waveform pattern. The neurostimulation may be delivered corresponding the repeated non-uniform waveform pattern, and the neural sensor is configured to sense at least a first neural response to neurostimulation corresponding to a first selected pulse in the repeated non-uniform waveform pattern. The controller may be configured to determine an average for the first neural response to neurostimulation corresponding to at least two instances of the first selected pulse in the repeated non-uniform waveform pattern and control the delivery of the neurostimulation from the neurostimulator based the average.

In Example 8, the subject matter of Example 7 may optionally be configured such that the average for the first neural response is a weighted average for the at least two instances.

In Example 9, the subject matter of Example 6 may optionally be configured such that the neural sensor is configured to sense at least a first neural response to neurostimulation corresponding to a first selected pulse in the non-uniform waveform pattern and a second neural response to neurostimulation corresponding to a second selected pulse in the non-uniform waveform pattern. The controller may be configured to determine a weighted average of the first and second neural responses and control the delivery of the neurostimulation based on the weighted average.

In Example 10, the subject matter of any one or more of Examples 1-9 may optionally be configured such that the controller is configured to control the delivery of the neurostimulation by modulating a mean for at least one pulse parameter based on the sensed neural signal.

In Example 11, the subject matter of any one or more of Examples 1-10 may optionally be configured such that the non-uniform waveform pattern has a variable pulse parameter with parameter values within a parameter range that includes a minimum parameter value and a maximum parameter value within the parameter range. The at least one sensing window may be automatically assigned to sense a first extrema neural response to the neurostimulation corresponding to the minimum parameter value and a second extrema neural response to the neurostimulation corresponding to the maximum parameter value. The controller may be configured to determine a desired neural response for the automatically assigned sensing window based on the first and second extrema neural responses, use a deviation between the sensed neural response and the desired neural response to control the delivery of the neurostimulation, and control the delivery of the neurostimulation based on the deviation by modulating at least one of a center, depth or period for the variable pulse parameter to provide the desired neural response.

In Example 12, the subject matter of any one or more of Examples 1-11 may optionally be configured such that the controller is configured to control the neurostimulator and the neural sensor to sweep neurostimulation through a plurality of parameter values to collect threshold neural response data at one or more threshold parameter values, designate threshold level specifications for the non-uniform waveform pattern based on the one or more threshold parameter values and the collected threshold neural response data, and control the neurostimulator to maintain the neurostimulation within the threshold level specifications using the collected threshold neural response data. The one or more threshold parameter values may include one or more of a perception threshold parameter value, a maximum comfort threshold parameter value, or a discomfort threshold parameter value.

In Example 13, the subject matter of any one or more of Examples 1-12 may optionally be configured such that the controller is configured to identify a meaningful epoch in the non-uniform waveform and assign the sensing window during the identified meaningful epoch.

In Example 14, the subject matter of any one or more of Examples 1-13 may optionally be configured such that the controller is configured to identify when an evoked potential or a local field potential change is expected and automatically assign the sensing window during a quiescent period within the non-uniform waveform pattern when the evoked potential or the change in field potentials is expected.

In Example 15, the subject matter of any one or more of Examples 1-14 may optionally be configured such that the controller is configured to determine a quiescent period within the non-uniform waveform pattern that is longer than a threshold period of time and automatically assign the sensing window to the quiescent period that is determined longer than the threshold.

Example 16 includes subject matter (such as a method, means for performing acts, machine readable medium including instructions that when performed by a machine cause the machine to perform acts, or an apparatus to perform). The subject matter may be include accessing a non-uniform waveform pattern for use to deliver neurostimulation, automatically assigning, based on the non-uniform waveform pattern, at least one sensing window for the non-uniform waveform pattern, delivering neurostimulation corresponding the non-uniform waveform pattern, sensing a neural signal during the at least one sensing window when the neurostimulation is being delivered, and controlling the delivery of the neurostimulation based on the sensed neural signal.

In Example 17, the subject matter of Example 16 may optionally be configured such that the non-uniform waveform pattern includes activation pulses that cause a neural response and sub-activation threshold pulses that do not cause the neural response. The automatically assigning the at least one sensing window may include determining at least one pulse in the non-uniform waveform pattern that corresponds to the neural response and assigning the at least one sensing window to sense the neural response.

In Example 18, the subject matter of Example 17 may optionally be configured such that the determining the at least one pulse in the non-uniform waveform pattern includes using a relationship between a neural activation and at least one pulse parameter for the at least one pulse to determine the at least one pulse in the non-uniform waveform pattern that corresponds to the neural response.

In Example 19, the subject matter of Example 18 may optionally be configured such that the relationship is between the neural activation and at least one of a pulse amplitude, a pulse frequency or a pulse width.

In Example 20, the subject matter of Example 17 may optionally be configured such that determining the at least one pulse in the non-uniform waveform pattern includes automatically determining a largest pulse amplitude in the non-uniform waveform pattern. The at least one sensing window may be automatically assigned to sense a neural response to the neurostimulation corresponding to the largest pulse amplitude in the non-uniform waveform pattern.

In Example 21, the subject matter of Example 17 may optionally be configured such that the determining the at least one pulse in the non-uniform waveform pattern includes automatically selecting multiple pulses in the non-uniform waveform pattern. The at least one sensing window may be assigned to sense a neural response to the neurostimulation corresponding to the automatically selected multiple pulses.

In Example 22, the subject matter of Example 17 may optionally be configured such that the non-uniform waveform pattern is repeated to provide multiple instances of each pulse in the non-uniform waveform pattern and the neurostimulation is delivered corresponding the repeated non-uniform waveform pattern. The sensing the neural signal may include sensing at least a first neural response to neurostimulation corresponding to a first selected pulse in the repeated non-uniform waveform pattern. The subject matter may include averaging the first neural response to neurostimulation corresponding to at least two instances of the first selected pulse in the repeated non-uniform waveform pattern.

In Example 23, the subject matter of Example 22 may optionally be configured such that the averaging the first neural response includes determining a weighted average of the first neural response corresponding to the at least two instances.

In Example 24, the subject matter of Example 21 may optionally be configured such that the sensing the neural signal includes sensing at least a first neural response to neurostimulation corresponding to a first selected pulse in the non-uniform waveform pattern and a second neural response to neurostimulation corresponding to a second selected pulse in the non-uniform waveform pattern. The subject matter may further include determining a weighted average of the first and second neural responses and controlling the delivery of the neurostimulation based on the weighted average.

In Example 25, the subject matter of Example 16 may optionally be configured such that the controlling the delivery of the neurostimulation based on the sensed neural signal includes modulating a mean for at least one pulse parameter based on the sensed neural signal.

In Example 26, the subject matter of Example 16 may optionally be configured such that the non-uniform waveform pattern has a variable pulse parameter with parameter values within a parameter range that includes a minimum parameter value and a maximum parameter value within the parameter range, and the at least one sensing window is automatically assigned to sense a first extrema neural response to the neurostimulation corresponding to the minimum parameter value and a second extrema neural response to the neurostimulation corresponding to the maximum parameter value. The subject matter may further include determining a desired neural response for the automatically assigned sensing window based on the first and second extrema neural responses and using a deviation between the sensed neural response and the desired neural response to control the delivery of the neurostimulation.

In Example 27, the subject matter of Example 26 may optionally be configured such that the controlling the delivery of the neurostimulation based on the deviation includes modulating at least one of a center, depth or period for the variable pulse parameter to provide the desired neural response.

In Example 28, the subject matter of Example 16 may optionally be configured such that sweeping neurostimulation through a plurality of parameter values to collect threshold neural response data at one or more threshold parameter values, designating threshold level specifications for the non-uniform waveform pattern based on the one or more threshold parameter values and the collected threshold neural response data, and maintaining the neurostimulation within the threshold level specifications, using the collected threshold neural response data.

In Example 29, the subject matter of Example 28 may optionally be configured such that the sweeping the neurostimulation includes sweeping past a perception threshold parameter value, a maximum comfort threshold parameter value, or a discomfort threshold parameter value.

In Example 30, the subject matter of Example 16 may optionally be configured to further include identifying a meaningful epoch in the non-uniform waveform, wherein the sensing window is automatically assigned during the identified meaningful epoch.

In Example 31, the subject matter of Example 16 may optionally be configured to further include identifying when an evoked potential or a local field potential change is expected, wherein the sensing window is automatically assigned during a quiescent period within the non-uniform waveform pattern when the evoked potential or the change in field potentials is expected.

In Example 32, the subject matter of Example 16 may optionally be configured to further include determining a quiescent period within the non-uniform waveform pattern that is longer than a threshold period of time. The sensing window may be automatically assigned to the quiescent period that is determined longer than the threshold.

In Example 33, the subject matter of Example 32 may optionally be configured such that the threshold period of time is a user-programmable period of time.

In Example 34, the subject matter of Example 16 may optionally be configured such that the automatically assigning includes automatically assigning a first sensing window in a first non-uniform waveform pattern and automatically assigning a second sensing window in a second non-uniform waveform pattern. The delivering neurostimulation may include delivering first neurostimulation corresponding to a first non-uniform waveform pattern at a first stimulation site and delivering second neurostimulation corresponding to a second non-uniform waveform pattern at a second stimulation site. The sensing the neural signal may include sensing a first neural response to the first neurostimulation within the first sensing window and sensing a second neural response to the second neurostimulation within the second sensing window. The controlling may include controlling delivery of at least one of the first neurostimulation or the second neurostimulation based on both of the first neural response and the second neural response.

Example 35 includes subject matter (such as a non-transitory machine-readable medium including instructions, which when executed by a machine, cause the machine to perform a method that include accessing a non-uniform waveform pattern for use to deliver neurostimulation, automatically assigning, based on the non-uniform waveform pattern, at least one sensing window for the non-uniform waveform pattern, delivering neurostimulation corresponding the non-uniform waveform pattern, sensing a neural signal during the at least one sensing window when the neurostimulation is being delivered, and controlling the delivery of the neurostimulation based on the sensed neural signal.

In further examples, the subject matter of Example 35 may be configured such that the method performed by the machine may include any of the subject matter recited in Examples 17-34.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an embodiment of a neurostimulation system.

FIG. 2 illustrates, by way of example and not limitation, an embodiment of a stimulation device and a lead system.

FIG. 3 illustrates, by way of example and not limitation, an embodiment of a programming device.

FIG. 4 illustrates, by way of example and not limitation, an implantable neurostimulation system and portions of an environment in which system may be used.

FIG. 5 illustrates, by way of example and not limitation, an embodiment of an implantable stimulator and lead(s) of an implantable neurostimulation system.

FIG. 6 illustrates, by way of example and not limitation, an embodiment of an external programming device of an implantable neurostimulation system.

FIG. 7 illustrates, by way of example and not limitation, an embodiment of a waveform composer.

FIG. 8 illustrates, by way of example and not limitation, a method that includes accessing a non-uniform waveform pattern for use to deliver neurostimulation and automatically assigning, based on the non-uniform waveform pattern, at least one sensing window for the non-uniform waveform pattern.

FIG. 9 illustrates, by way of example and not limitation, a system according to various embodiments.

FIG. 10 illustrates, by way of example and not limitation, a neural activation curve plotting a sensed neural signal against a neurostimulation parameter.

FIG. 11 illustrates, by way of example and not limitation, a non-uniform waveform pattern which may include amplitudes corresponding to neurostimulation below the ECAP threshold and amplitudes corresponding to neurostimulation above the ECAP threshold.

FIG. 12 illustrates, by way of example and not limitation, a neural activation curve plotting a value for an ECAP feature against a neurostimulation frequency.

FIG. 13 illustrates, by way of example and not limitation, a neural activation curve plotting a value for an ECAP feature against a neurostimulation pulse width.

FIG. 14 illustrates, by way of example and not limitation, the use of extrema pulses for determining sense events and modifying the stimulation pattern based on recorded and target sense events.

FIG. 15 illustrates, by way of example and not limitation, a clinical workflow using extrema pulses to guide adaptation of patterns.

FIG. 16 illustrates, by way of example and not limitation, quiescent period sensing in a non-uniform waveform parameter.

FIG. 17 illustrates, by way of example and not limitation, paired pulse sensing.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments control when, with respect to a non-uniform waveform pattern used to deliver neurostimulation, to sense neural signals to appropriately control neurostimulation. A more accurate comparison may be performed between sensed neural signals if they consistently reflect a response to similar stimulation events. Various embodiments provided herein automatically determine how sensing windows are assigned when neurostimulation is delivered according to a non-uniform waveform stimulation pattern. For example, some embodiments assign sensing windows by determining the extrema of fiber activation profiles. By way of example and not limitation, fiber activation profiles may be analyzed using evoked compound action potential (ECAP) morphologies. Thus, extrema for ECAP morphologies may be analyzed to determine how sensing windows are assigned when neurostimulation is delivered according to a non-uniform waveform stimulation pattern. Some embodiments may assign sensing windows by tracking the average fiber activation profile. Some embodiments may assign sensing windows by determining a neurostimulation pulse that is most likely to produce a detectable ECAP. Some embodiments may assign sensing windows based on a type or characteristic of the non-uniform waveform pattern. The waveform pattern may be characterized based on the amplitude, pulse width, and pulse-to-pulse intervals. A waveform pattern may be characterized using a minimum and/or maximum pulse-to-pulse interval in the non-uniform waveform pattern, and/or the number of pulses delivered within a time interval. For example, a waveform pattern may be characterized by the number of pulses within the time interval that are above a neural response threshold (e.g., ECAP feature(s)). Some embodiments may assign sensing window(s) to select pulses (e.g., also referred to as “sensing pulses”) within the non-uniform waveform pattern. The selection of the sensing pulses may be based on a neural activation curve.

Advancements in neuroscience and neurostimulation research have led to a demand for using complex and/or individually optimized patterns of neurostimulation pulses for various types of therapies. By way of example and not limitation, some systems may enable a user to define a pattern of neurostimulation pulses, which includes defining waveforms being the building blocks of the pattern. For example, a user interface may allow a user to define a potentially very complex patterns of neurostimulation pulses by creating and editing graphical representations of relatively simple individual building blocks for each of the patterns. The waveforms may include pulses, bursts of pulses, trains of bursts, and sequences of pulses, bursts, and trains. The patterns of neurostimulation pulses are not limited to waveforms predefined at the time of manufacturing.

A combination of hardware and software may be designed to provide users such as researchers, physicians or other caregivers, or neurostimulation device makers with ability to non-uniform waveform patterns in an effort to increase therapeutic efficacy and/or patient satisfaction for neurostimulation therapies, including but not being limited to deep brain stimulation (DBS), spinal cord stimulation (SCS), peripheral nerve stimulation (PNS), and vagus nerve stimulation (VNS). Various embodiments described herein control when, with respect to a non-uniform waveform pattern used to deliver neurostimulation, to sense neural signals. This may be particularly useful to control the neurostimulation and/or determine the effectiveness of a neurostimulation therapy. Some embodiments allow the user to determine when the sense window(s) occur with respect to the non-uniform waveform pattern, and some embodiments automatically determine when to implement sense window(s) for a defined non-uniform waveform pattern.

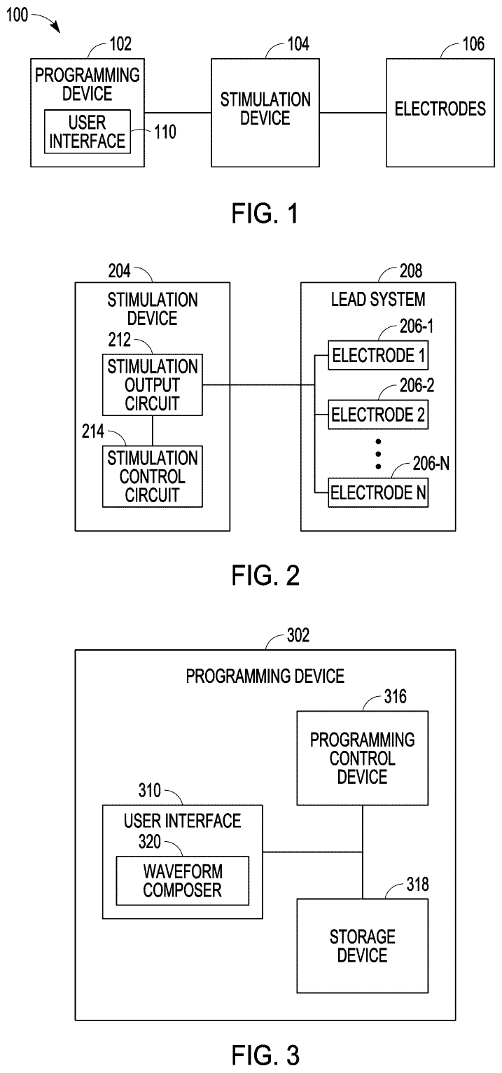

FIG. 1 illustrates, by way of example and not limitation, an embodiment of a neurostimulation system 100. The system 100 may include electrodes 106, a stimulation device 104, and a programming device 102. The electrodes 106 may be configured to be placed on or near one or more neural targets in a patient. The stimulation device 104 may be configured to be electrically connected to the electrodes 106 and deliver neurostimulation energy, such as in the form of electrical pulses, to the one or more neural targets though the electrodes 106. The delivery of the neurostimulation may be controlled by using a plurality of stimulation parameters such as stimulation parameters specifying a pattern of the electrical pulses and a selection of electrodes through which each of the electrical pulses is delivered. In various embodiments, at least some parameters of the plurality of stimulation parameters may be programmable by a user such as a physician or other caregiver who treats the patient using the system 100. The programming device 102 may provide the user with accessibility to the user-programmable parameters and may be configured to be communicatively coupled to stimulation device via a wired or wireless link.

Some neurostimulation systems may be configured to use predefined nonuniform patterns to deliver the neurostimulation, and some neurostimulation systems may be configured for a user to develop the nonuniform patterns. In various embodiments, the programming device 102 may include a user interface that allows the user to set and/or adjust values of the user-programmable parameters by creating and/or editing graphical representations of various waveforms. Such waveforms may include, for example, the waveform of a pattern of neurostimulation pulses to be delivered to the patient as well as waveform building blocks that can be used in the pattern of neurostimulation pulses. Examples of such waveform building blocks include pulses, bursts each including a group of the pulses, trains each including a group of the bursts, and sequences each including a group of the pulses, bursts, and trains, as further discussed below. In various embodiments, programming device 102 allows the user to edit existing waveform building blocks, create new waveform building blocks, import waveform building blocks created by other users, and/or export waveform building blocks to be used by other users. The user may also be allowed to define an electrode selection specific to each waveform building block. In the illustrated embodiment, the user interface includes a user interface 110. In various embodiments, user interface 110 may include a GUI or any other type of user interface accommodating various functions including waveform composition as discussed in this document.

FIG. 2 illustrates, by way of example and not limitation, an embodiment of a stimulation device 204 and a lead system 208, such as may be implemented in neurostimulation system 100. The stimulation device 204 may represent an embodiment of the stimulation device 104 in FIG. 1 and may include a stimulation output circuit 212 and a stimulation control circuit 214. The stimulation output circuit 212 may produce and deliver neurostimulation pulses. The stimulation control circuit 214 may control the delivery of the neurostimulation pulses using the plurality of stimulation parameters which specifies a pattern of the neurostimulation pulses. The lead system 208 may include one or more leads each configured to be electrically connected to stimulation device 204 and a plurality of electrodes 206 distributed in the one or more leads. The plurality of electrodes 206 may include electrode 206-1, electrode 206-2, . . . electrode 206-N, each a single electrically conductive contact providing for an electrical interface between stimulation output circuit 212 and tissue of the patient, where N≥2. The neurostimulation pulses may each delivered from the stimulation output circuit 212 through a set of electrodes selected from the electrodes 206. The number of leads and the number of electrodes on each lead depend on, for example, the distribution of target(s) of the neurostimulation and the desired electric field(s) at each target. In a nonlimiting example, the lead system 208 may include 2 leads each having 8 electrodes.

FIG. 3 illustrates, by way of example and not limitation, an embodiment of a programming device 302. The programming device 302 may be implemented in the neurostimulation system 100 and may represents an embodiment of programming device 102. The programming device 302 may include a storage device 318, a programming control circuit 316, and a user interface 310. The storage device 318 may store a plurality of waveform building blocks. The programming control circuit 316 may generate the plurality of stimulation parameters that controls the delivery of the neurostimulation pulses according to the pattern of the neurostimulation pulses. The user interface 310 may represent an embodiment of a user interface 110 and allows the user to compose the waveform building blocks and compose the pattern of the neurostimulation pulses.

In various embodiments, the user interface 310 may include a waveform composer 320 that allows the user to manage the waveform building blocks, including creating and importing waveform building blocks to be added to the waveform building blocks stored in storage device 318, exporting waveform building blocks selected from the waveform building blocks stored in storage device 318, and editing each of the waveform building blocks. For example, the user interface 310 may include a GUI that allows for graphical editing of each of the waveform building blocks. The waveform composer 320 allows the user to compose the pattern of neurostimulation pulses to be delivered using stimulation device 104 using waveform building blocks such as pulses, bursts each including a group of the pulses, trains each including a group of the bursts, and/or sequences each including a group of the pulses, bursts, and trains.

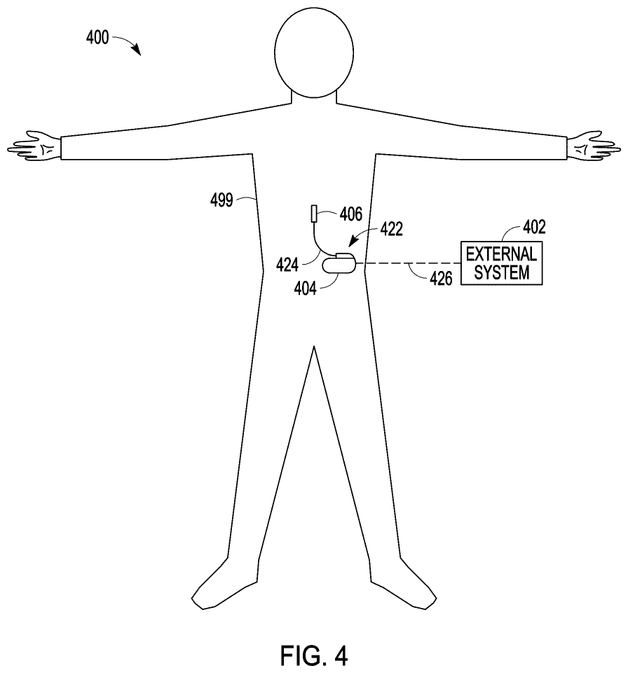

FIG. 4 illustrates, by way of example and not limitation, an implantable neurostimulation system 400 and portions of an environment in which system 400 may be used. The system 400 may include an implantable system 422, an external system 402, and a telemetry link 426 providing for wireless communication between the implantable system 422 and the external system 402.

The implantable system 422 may include an implantable stimulator (also referred to as an implantable pulse generator, or IPG) 404, a lead system 424, and electrodes 406, which may represent an embodiment of the stimulation device 204, the lead system 208, and the electrodes 206, respectively. The external system 402 may represent an embodiment of the programming device 302. In various embodiments, the external system 402 may include one or more external (non-implantable) devices each allowing the user and/or the patient to communicate with the implantable system 422. In some embodiments, the external system 402 may include a programming device intended for the user to initialize and adjust settings for implantable stimulator 404 and a remote control device intended for use by the patient. For example, the remote control device may allow the patient to turn implantable stimulator 404 on and off and/or adjust certain patient-programmable parameters of the plurality of stimulation parameters. In some embodiments, the external system may include cloud computing, fog computing and/or edge computing, which may in some examples be used to create the non-uniform waveform which may include the sensing window(s), create models used to determine when to perform the neural sensing to provide desired therapeutic efficacy, how to modify the neurostimulation parameters in response to sensed parameters, and/or process the sensed neural signals.

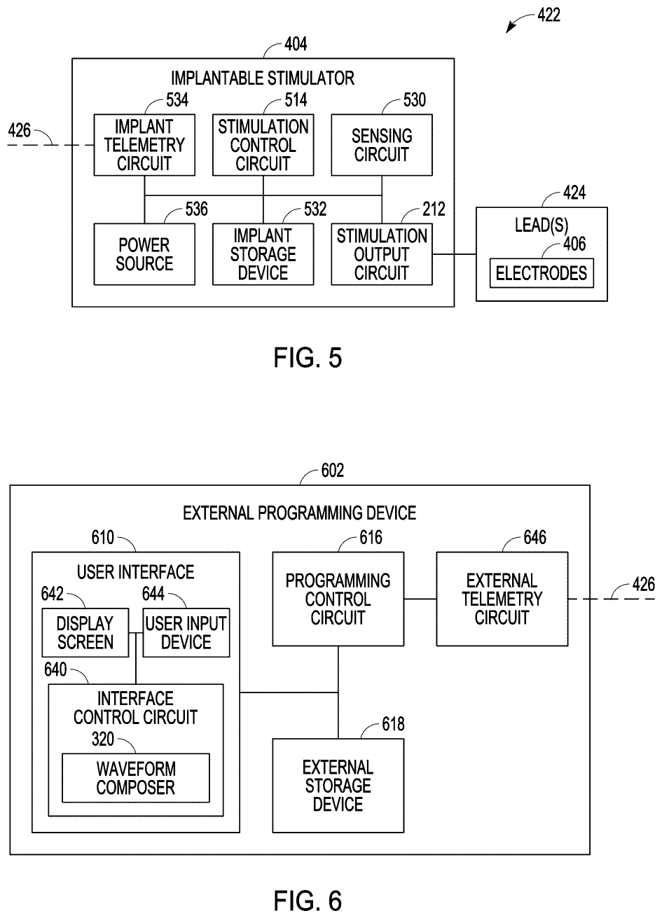

FIG. 5 illustrates, by way of example and not limitation, an embodiment of an

implantable stimulator 404 and one or more leads 424 of an implantable neurostimulation system. The implantable stimulator 404 may include a sensing circuit 530, stimulation output circuit 212, a stimulation control circuit 514, an implant storage device 532, an implant telemetry circuit 534, and a power source 536. The sensing circuit 530 is configured for use in sensing one or more physiological signals for purposes of patient monitoring and/or feedback control of the neurostimulation. Examples of the one or more physiological signals includes neural and other signals each indicative of a condition of the patient that is treated by the neurostimulation and/or a response of the patient to the delivery of the neurostimulation. The stimulation output circuit 212 may be electrically connected to electrodes 406 through the lead 424 and may deliver each of the neurostimulation pulses through a set of electrodes selected from the electrodes 406. The stimulation control circuit 514 may represent an embodiment of stimulation control circuit 214 and may control the delivery of the neurostimulation pulses using the plurality of stimulation parameters specifying the pattern of the neurostimulation pulses. The stimulation control circuit 514 may control the delivery of the neurostimulation pulses using the one or more sensed physiological signals. The implant telemetry circuit 534 provides implantable stimulator 404 with wireless communication with another device such as a device of external system 402, including receiving values of the plurality of stimulation parameters from external system 402. The implant storage device 532 stores values of the plurality of stimulation parameters. The power source 536 provides implantable stimulator 404 with energy for its operation. The power source 536 may include a battery. For example, the power source 536 may include a rechargeable battery and a battery charging circuit for charging the rechargeable battery. The implant telemetry circuit 534 may also function as a power receiver that receives power transmitted from the external system 402 through an inductive couple.

FIG. 6 illustrates, by way of example and not limitation, an embodiment of an external programming device 602 of an implantable neurostimulation system. The external programming device 602 may represent an embodiment of programming device 302, and may include an external telemetry circuit 646, an external storage device 618, a programming control circuit 616, and a user interface 610. The external telemetry circuit 646 may provide the external programming device 602 with wireless communication with another device such as implantable stimulator 404 via telemetry link 426, including transmitting the plurality of stimulation parameters to implantable stimulator 404. The external telemetry circuit 646 also may transmit power to implantable stimulator 404 through the inductive couple.

The external storage device 618 may store a plurality of waveform building blocks each selectable for use as a portion of the pattern of the neurostimulation pulses. Each waveform building block of the plurality of waveform building blocks may include one or more pulses of the neurostimulation pulses and may include one or more other waveform building blocks of the plurality of waveform building blocks. Examples of such waveforms include pulses, bursts each including a group of the pulses, trains each including a group of the bursts, and sequences each including a group of the pulses, bursts, and trains. The external storage device 618 also may store a plurality of stimulation fields. Each waveform building block of the plurality of waveform building blocks may be associated with one or more fields of the plurality of stimulation fields. Each field of the plurality of stimulation fields may be defined by one or more electrodes of the plurality of electrodes through which a pulse of the neurostimulation pulses is delivered and a current distribution of the pulse over the one or more electrodes.

The programming control circuit 616 may generate the plurality of stimulation parameters, which is to be transmitted to implantable stimulator 404, according to the pattern of the neurostimulation pulses. The pattern may be defined using one or more waveform building blocks selected from the plurality of waveform building blocks stored in external storage device 618. The programming control circuit 616 may check values of the plurality of stimulation parameters against safety rules to limit these values within constraints of the safety rules. In one embodiment, the safety rules are heuristic rules.

The user interface 610 may allow the user to define the pattern of neurostimulation pulses and perform various other monitoring and programming tasks. The interface control circuit 640 controls the operation of user interface 610 including responding to various inputs received by user input device 644 and defining the one or more stimulation waveforms. The interface control circuit 640 may include the waveform composer 320.

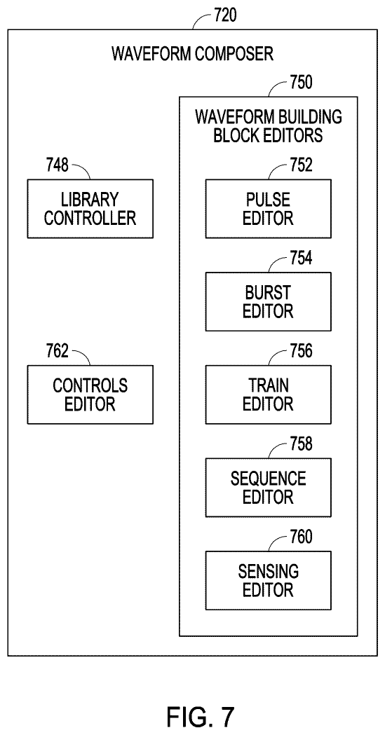

FIG. 7 illustrates, by way of example and not limitation, an embodiment of a waveform composer 720. The waveform composer 720 allows for composition of one of more waveform building blocks of the plurality of waveform building blocks stored in the external storage device 618 and composition of the pattern of the neurostimulation pulses using one of more stimulation building blocks selected from the plurality of waveform building blocks stored in the external storage device 618. In the illustrated embodiment, the waveform composer includes a library controller 748 and a plurality of waveform building block editors 750. The library controller 748 displays a library management area on display screen 642 used to manage the waveform building blocks stored in external storage device 618. The waveform building block editors 750 may each display a composition area for a type of the waveform building blocks of a plurality of types of the waveform building blocks on the display screen 642. The displayed composition area allows the user to compose each waveform building block of the type of the waveform building blocks.

The waveform building block editors 750 may include an editor for each type of the plurality of types of waveform building blocks. In the illustrated embodiment, the waveform building block editors 750 include a pulse editor 752, a burst editor 754, a train editor 756, a sequence editor 758, and a sensing editor 760.

The pulse editor 752 displays a pulse composition area on display screen 642 in response to a user selection for access to the pulse editor. The pulse composition area allows the user to compose a pulse of the waveform building blocks. A pulse composition area may allow the user to edit a pulse selected from the waveform building blocks stored in external storage device 618 and to create a new pulse to be added to the waveform building blocks stored in external storage device 618. The pulse composition area displays a graphical representation of the pulse being edited or created and a slider for shifting, expanding, or contracting a timeline of the graphical representation of the pulse. The pulse composition area allows the user to select a pulse editing mode from a plurality of pulse editing modes, such as by displaying a pull down menu listing the plurality of pulse editing modes as illustrated. Examples of the pulse editing modes include, but are not limited to, a guided mode, a free form mode, and a draw mode. Under the guided mode, values of parameters defining the pulse are displayed, and the user is allowed to edit the pulse by adjusting the displayed values of the parameters. Under the free form mode, the user is allowed to edit the pulse by graphically modifying the displayed graphical representation of the pulse. Under the draw mode, the user is allowed to sketch a waveform for the pulse. In response to a selection of automatic charge balancing by the user, pulse editor 742 can automatically modify the pulse for charge balancing. The pulse editor may be configured to insert and/or modify one or more sensing pulses. The sensing pulse(s) may be configured to generate a neural response, and sensing window(s) may be configured for sensing a neural signal that corresponds to the neural response.

The burst editor 754 displays a burst composition area on the display screen 642 in response to a user selection for access to the burst editor. The burst composition area allows the user to compose a burst of the waveform building blocks. The burst composition area allows the user to edit a burst selected from the waveform building blocks stored in the external storage device 618 or to create a new burst to be added to the waveform building blocks stored in the external storage device 618. The burst composition area displays a preview of a waveform of the burst and allows for saving of modified waveform of the burst. The burst composition area allows the user to select options for editing each of the characteristics of the burst, such as duration, location (location in the body of the patient to which the burst is applied, i.e., electrode configuration), pulse frequency, pulse type, and pulse amplitude.

The train editor 746 displays a train composition area on the display screen 642 in response to a user selection for access to the train editor. The train composition area allows the user to compose a train of the waveform building blocks. The train composition area allows the user to edit a train selected from the waveform building blocks stored in the external storage device 618 or to create a new train to be added to the waveform building blocks stored in the external storage device 618. The train composition area displays a preview of a waveform of the train and allows for saving of modified waveform of the train. The train composition area allows the user to select options for editing each of the characteristics of the train, such as duration, burst location, burst frequency, train configuration, and burst amplitude.

The sequence editor 748 displays a sequence composition area on the display screen 642 in response to a user selection for access to the sequence editor. The sequence composition area allows the user to compose a sequence of the waveform building blocks. The sequence composition area allows the user to edit a sequence selected from the waveform building blocks stored in external storage device 618 or to create a new sequence to be added to the waveform building blocks stored in the external storage device 618. The sequence composition area displays a preview of a waveform of the sequence and allows for saving of modified waveform of the sequence. The sequence composition area allows for selection of a sequence editing option from a plurality of sequence editing modes, allows for addition and deletion of waveform building blocks (sequence components) in the sequence, and allows for simple editing of the waveform building blocks within the sequence composition area. The sensing editor 760 may be configured for the user to select or modify sensing window(s) used for sensing a neural signal that corresponds to the neural response. For example, the user may select or modify rules for automatically identify or determine when the sensing window(s) occur with respect to the non-uniform pattern.

The sensing editor 760 may be configured to work with the pulse editor to insert and/or modify one or more sensing pulses. The sensing pulse(s) may be configured to generate a neural response, and sensing window(s) may be configured for sensing a neural signal that corresponds to the neural response.

The controls editor 762 displays a controls area on display screen 642 in response to a user command. The controls area allows the user to edit pulse parameters used for a waveform building block.

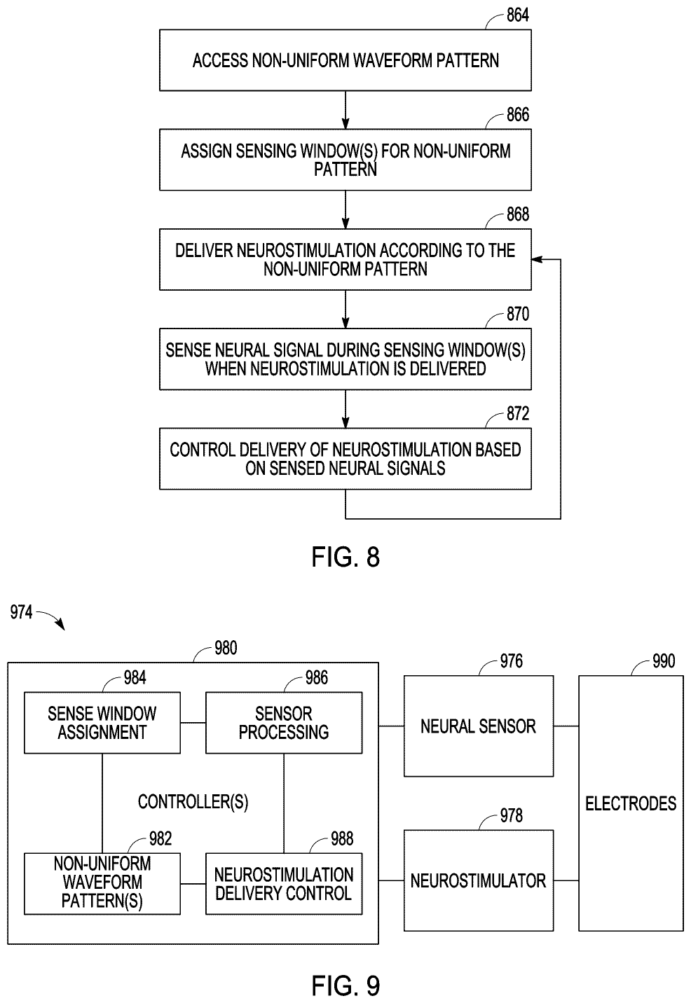

FIG. 8 illustrates, by way of example and not limitation, a method that includes accessing a non-uniform waveform pattern for use to deliver neurostimulation 864 and automatically assigning, based on the non-uniform waveform pattern, at least one sensing window for the non-uniform waveform pattern 866. The non-uniform waveform pattern may be created by a user, such as using a waveform composer, may be a predefined pattern such as may be stored in and retrieved from a local or remote memory, or may be created or modified using machine learning or other artificial intelligence. The illustrated method may further include delivering neurostimulation corresponding the non-uniform waveform pattern 868. Thus, for example, a neurostimulation waveform may include pulses that closely correspond to the pulses in the non-uniform waveform pattern. The method may include sensing a neural signal during the at least one sensing window when the neurostimulation is being delivered 870 and controlling the delivery of the neurostimulation based on the sensed neural signal 872.

FIG. 9 illustrates, by way of example and not limitation, a system according to various embodiments. The system 974 may include a neural sensor 976 configured to sense a neural signal, a neurostimulator 978 and at least one controller 980. The neural sensor 976 may be configured to sense evoked compound action potentials (ECAPs) or local field potentials. Other signals may be detected such as spinograms or evoked resonant neural activity (ERNA). The controller(s) 980 configured to automatically assign, based on the non-uniform waveform pattern 982, a sensing window for the non-uniform waveform pattern 984, control the neural sensor 976 to sense the neural signal during the sensing window when the neurostimulation is delivered 986, and control the delivery of the neurostimulation from the neurostimulator 978 based on the sensed neural signal 988. All the controller functions may be within a single device or may be distributed among two or more devices. For example, the functions associated with the window assignment and the patterns may be performed in an external device (e.g., a programmer) and/or in an implantable device (e.g., neurostimulator). Some of the function(s) may be performed using cloud computing, fog computing and/or edge computing. Cloud computing may include a network of devices or servers connected over the Internet. Cloud computing may have very large storage space and processing capabilities. However, cloud computing can have higher latencies. Fog computing occurs physically closer to the end user compared to centralized data centers. The infrastructure of fog computing may connect end devices with central servers in the cloud. Fog computing may provide lower latency for quicker responses and may use other communication technology other than the Internet. Edge computing is done at the device level. The processing for different functions may be distributed over multiple devices and may be distributed over edge computing, fog computing and cloud computing. Electrodes 990 may be used by the neural sensor 976 and/or the neurostimulator 978. For example, the electrodes 990 may be distributed on one or more leads to provide a plurality of electrodes within an electrode array

The non-uniform waveform pattern may include activation pulses that cause a neural response (e.g., an ECAP feature) and sub-activation threshold pulses that do not cause the neural response. The sensing window(s) may be automatically assigned by determining at least one pulse in the non-uniform waveform pattern that corresponds to the neural response and assigning the at least one sensing window to sense the neural response. A relationship between a neural activation and at least one pulse parameter for the at least one pulse may be used to determine the at least one pulse in the non-uniform waveform pattern that corresponds to the neural response. The relationship may be between the neural activation and at least one of a pulse amplitude, a pulse frequency or a pulse width. A largest pulse amplitude in the non-uniform waveform pattern may be automatically determined. The sensing window(s) may be automatically assigned to sense a neural response to the neurostimulation corresponding to the largest pulse amplitude in the non-uniform waveform pattern. Multiple pulses in the non-uniform waveform pattern may be automatically selected. The sensing window(s) may be assigned to sense a neural response to the neurostimulation corresponding to the automatically selected multiple pulses. The non-uniform waveform pattern may be repeated to provide multiple instances of each pulse in the non-uniform waveform pattern and the neurostimulation may be delivered corresponding the repeated non-uniform waveform pattern. The neural signal may be sensed by sensing at least a first neural response to neurostimulation corresponding to a first selected pulse in the repeated non-uniform waveform pattern. The subject matter may include averaging the first neural response to neurostimulation corresponding to at least two instances of the first selected pulse in the repeated non-uniform waveform pattern. The first neural response may be averaged by determining a weighted average of the first neural response corresponding to the at least two instances.

The neural signal may be sensed by sensing at least a first neural response to neurostimulation corresponding to a first selected pulse (e.g., 1198A) in the non-uniform waveform pattern and a second neural response to neurostimulation corresponding to a second selected pulse (e.g., 1198B, 1198C) in the non-uniform waveform pattern. A weighted average of the neural responses may be determined and the delivery of the neurostimulation may be controlled based on the weighted average. The delivery of the neurostimulation may be controlled based on the sensed neural signal by modulating a mean for at least one pulse parameter based on the sensed neural signal.

A meaningful epoch in the non-uniform waveform may be identified, wherein the sensing window is automatically assigned during the identified meaningful epoch. The method may include identifying when an evoked potential or a local field potential change is expected. The sensing window may be automatically assigned during a quiescent period within the non-uniform waveform pattern when the evoked potential or the change in field potentials is expected. The method may include determining a quiescent period within the non-uniform waveform pattern that is longer than a threshold period of time. The sensing window may be automatically assigned to the quiescent period that is determined longer than the threshold. The threshold period of time may be a user-programmable period of time. The automatically assigning may include automatically assigning a first sensing window in a first non-uniform waveform pattern and automatically assigning a second sensing window in a second non-uniform waveform pattern. The delivering neurostimulation may include delivering first neurostimulation corresponding to a first non-uniform waveform pattern at a first stimulation site and delivering second neurostimulation corresponding to a second non-uniform waveform pattern at a second stimulation site. The sensing the neural signal may include sensing a first neural response to the first neurostimulation within the first sensing window and sensing a second neural response to the second neurostimulation within the second sensing window. Delivery of at least one of the first neurostimulation or the second neurostimulation may be controlled based on both of the first neural response and the second neural response.

The neurostimulator may be configured to access a non-uniform waveform pattern and deliver the neurostimulation corresponding to the stored non-uniform waveform pattern. The non-uniform waveform pattern may include activation pulses that cause a neural response and sub-activation threshold pulses that do not cause the neural response. The controller may be configured to automatically assign the sensing window by determining at least one pulse in the non-uniform waveform pattern that corresponds to a neural activation and assigning the at least one sensing window to sense the neural response to the neural activation. The neurostimulation may be delivered using active ones of the electrodes. The controller(s) 980 may be configured to automatically assign, based on the non-uniform waveform pattern, a sensing window for the non-uniform waveform pattern, control the neural sensor to sense the neural signal during the sensing window when the neurostimulation is delivered, and control the delivery of the neurostimulation from the neurostimulator based on the sensed neural signal. The controller may be configured to determine the pulse(s) in the non-uniform waveform pattern using a relationship between a neural activation and at least one of a pulse amplitude, a pulse frequency or a pulse width for the pulse(s). The controller may be configured to determine the pulse(s) by automatically determining a largest pulse amplitude in the non-uniform waveform pattern, and the sensing window is automatically assigned to sense a neural response to the neurostimulation corresponding to the largest pulse amplitude in the non-uniform waveform pattern. The controller(s) may be configured to automatically select multiple pulses in the non-uniform waveform pattern, and the sensing window may be assigned to sense a neural response to the neurostimulation corresponding to the automatically selected multiple pulses.

The non-uniform waveform pattern may be repeated to provide multiple instances of each pulse in the non-uniform waveform pattern. The neurostimulation may be delivered corresponding the repeated non-uniform waveform pattern, and the neural sensor may be configured to sense at least a first neural response to neurostimulation corresponding to a first selected pulse in the repeated non-uniform waveform pattern. The controller may be configured to determine an average for the first neural response to neurostimulation corresponding to at least two instances of the first selected pulse in the repeated non-uniform waveform pattern and control the delivery of the neurostimulation from the neurostimulator based the average. The average for the first neural response may be a weighted average for the at least two instances.

The neural sensor may be configured to sense at least a first neural response to neurostimulation corresponding to a first selected pulse in the non-uniform waveform pattern and a second neural response to neurostimulation corresponding to a second selected pulse in the non-uniform waveform pattern. The controller may be configured to determine a weighted average of the first and second neural responses and control the delivery of the neurostimulation based on the weighted average. The controller may be configured to control the delivery of the neurostimulation by modulating a mean for at least one pulse parameter based on the sensed neural signal.

The non-uniform waveform pattern may have a variable pulse parameter with parameter values within a parameter range that includes a minimum parameter value and a maximum parameter value within the parameter range. The at least one sensing window may be automatically assigned to sense a first extrema neural response to the neurostimulation corresponding to the minimum parameter value and a second extrema neural response to the neurostimulation corresponding to the maximum parameter value. The controller may be configured to determine a desired neural response for the automatically assigned sensing window based on the first and second extrema neural responses, use a deviation between the sensed neural response and the desired neural response to control the delivery of the neurostimulation, and control the delivery of the neurostimulation based on the deviation by modulating at least one of a center, depth or period for the variable pulse parameter to provide the desired neural response.

The controller may be configured to control the neurostimulator and the neural sensor to sweep neurostimulation through a plurality of parameter values to collect threshold neural response data at one or more threshold parameter values, designate threshold level specifications for the non-uniform waveform pattern based on the one or more threshold parameter values and the collected threshold neural response data, and control the neurostimulator to maintain the neurostimulation within the threshold level specifications using the collected threshold neural response data. The threshold parameter value(s) may include one or more of a perception threshold parameter value, a maximum comfort threshold parameter value, or a discomfort threshold parameter value.

The controller may be configured to identify a meaningful epoch in the non-uniform waveform and assign the sensing window during the identified meaningful epoch. The controller may be configured to identify when an evoked potential or a local field potential change is expected and automatically assign the sensing window during a quiescent period within the non-uniform waveform pattern when the evoked potential or the change in field potentials is expected. The controller may be configured to determine a quiescent period within the non-uniform waveform pattern that is longer than a threshold period of time and automatically assign the sensing window to the quiescent period that is determined longer than the threshold.

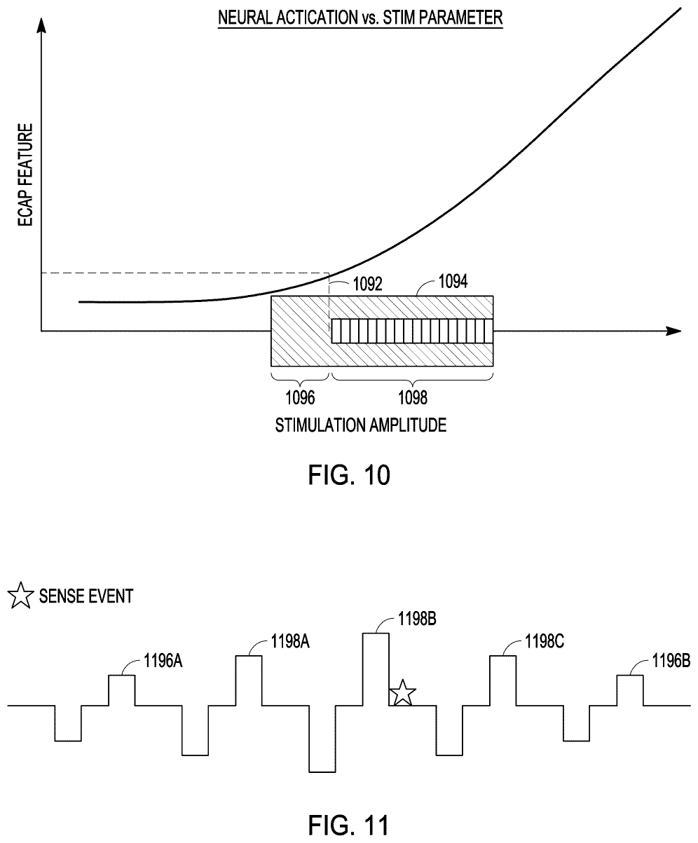

FIG. 10 illustrates, by way of example and not limitation, a neural activation curve plotting a sensed neural signal against a neurostimulation parameter. More particularly, the neural activation curve in FIG. 10 plots a value for an ECAP feature against a neurostimulation amplitude. FIG. 10 also illustrates an ECAP threshold 1092. The ECAP threshold 1092 refers to a certain level of neural activation that is needed to generate an ECAP feature. As illustrated, this level of neural activation needed to generate the ECAP feature corresponds to a threshold neurostimulation amplitude. Thus, in an example with a given set of stimulation parameters where only the amplitude is modified, the threshold neurostimulation amplitude is the threshold at which the neurostimulation delivered according to the given set of stimulation parameters generates ECAPs with the ECAP feature. All amplitudes included in the non-uniform waveform pattern are illustrated by indicator 1094, which includes a subset of amplitudes 1096 less than the threshold neurostimulation amplitude such that the corresponding neurostimulation pulses at these smaller amplitudes do not generate ECAPs with the ECAP feature and further includes a subset of amplitudes 1098 larger than the threshold neurostimulation amplitude such that the corresponding neurostimulation pulses at these larger amplitudes generate ECAPs with the ECAP feature.

FIG. 11 illustrates, by way of example and not limitation, a non-uniform waveform pattern which may include amplitudes (e.g., pulses 1196A and 1196B) corresponding to neurostimulation below the ECAP threshold and amplitudes (1198A, 1198B and 1198C) corresponding to neurostimulation above the ECAP threshold. According to some embodiments, sensing windows may only be assigned to those pulses that correspond to neurostimulation pulses that are above the neural response. The sensing pulses (pulses associated with a sensing event) may be selected to only be one or more pulses from those pulses that are above the neural response threshold (e.g., 1198A, 1198B, 1198C) and thus cause neural responses, allowing adaptive algorithms to function well because the sensed response is responsive to the neurostimulation pulse and quickly responds to changes in the neurostimulation parameter. By way of example and not limitation, an external device such as a clinical programmer may select the sensing pulse in the background. A pulse within the non-uniform waveform pattern that corresponds to the largest evoked response (e.g., 1198B) may be used as a default sensing pulse selection. Some embodiments may enable user-selection of the sensing pulse using any pulse(s) within the non-uniform waveform pattern that corresponds to a neurostimulation pulse that is over a neural response threshold such that the pulse can cause an evoked response.

Some embodiments may assign sensing window(s) based on a sequence of multiple pulses. For example, the sensing window(s) may be assigned based on two successive pulses (e.g., 1198A, 1198B) that are over the neural response threshold. Other sequence(s) may be used, including more complicated sequences involving more pulses and various timing for the pulses. This may allow information to be obtained more quickly and may be used where there are long sequences and/or noisy signals. For example, the response for each pulse in sequence may be sensed, and the sensed responses may be averaged or weighted. The pulse sequence scale may be modulated based on the sensed signals.

The neural activation curve in FIG. 10 is for a modulated pulse amplitude. Other neural activation curves may be used such as curve for a modulated pulse width or a modulate frequency. Frequency modulation, in particular, is expected to have a different relationship with respect to ECAP threshold than amplitude and pulse width.

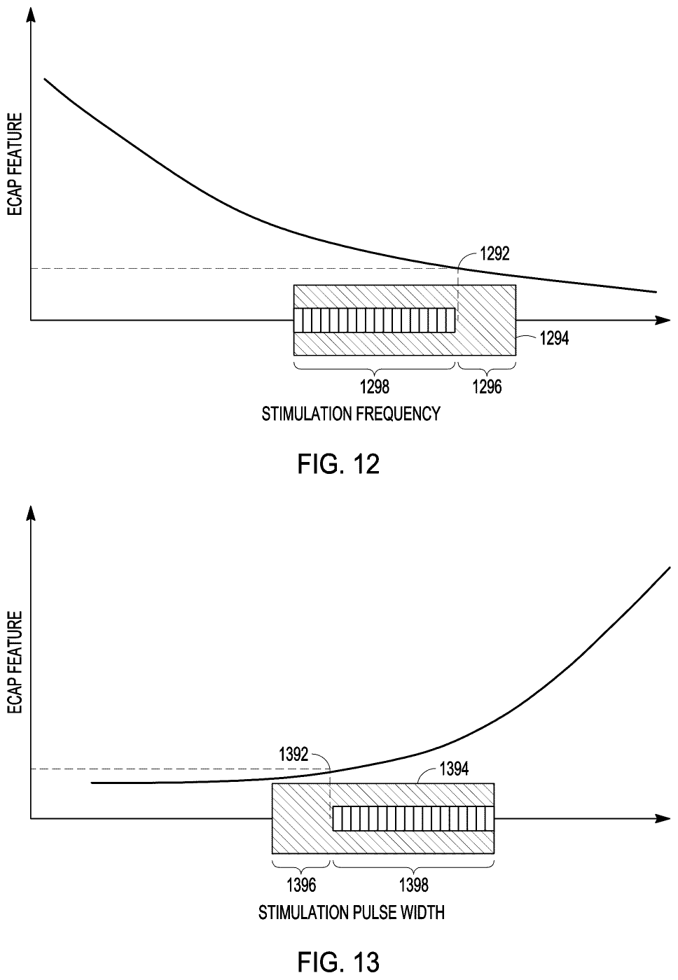

FIG. 12 illustrates, by way of example and not limitation, a neural activation curve plotting a value for an ECAP feature against a neurostimulation frequency. The ECAP threshold 1292 refers to a certain level of neural activation that is needed to generate an ECAP feature, and this level of neural activation corresponds to a threshold neurostimulation frequency. Thus, in an example with a given set of stimulation parameters where only the frequency is modified, the threshold neurostimulation frequency is the threshold below which the neurostimulation delivered according to the given set of stimulation parameters generates ECAPs with the ECAP feature. All frequencies included in the non-uniform waveform pattern are illustrated by indicator 1294, which includes a subset of frequencies 1296 more than the threshold neurostimulation amplitude such that the corresponding neurostimulation pulses at these larger frequencies do not generate ECAPs with the ECAP feature and further includes a subset of frequencies 1298 smaller than the threshold neurostimulation frequency such that the corresponding neurostimulation pulses at these smaller frequencies generate ECAPs with the ECAP feature.

FIG. 13 illustrates, by way of example and not limitation, a neural activation curve plotting a value for an ECAP feature against a neurostimulation pulse width. The ECAP threshold 1392 refers to a certain level of neural activation that is needed to generate an ECAP feature, and this level of neural activation corresponds to a threshold neurostimulation pulse width. Thus, in an example with a given set of stimulation parameters where only the pulse width is modified, the threshold neurostimulation pulse width is the threshold above which the neurostimulation delivered according to the given set of stimulation parameters generates ECAPs with the ECAP feature. All pulse widths included in the non-uniform waveform pattern are illustrated by indicator 1394, which includes a subset of frequencies 1396 less than the threshold neurostimulation pulse width such that the corresponding neurostimulation pulses at these small pulse widths do not generate ECAPs with the ECAP feature and further includes a subset of frequencies 1398 larger than the threshold neurostimulation pulse width such that the corresponding neurostimulation pulses at these larger pulse widths generate ECAPs with the ECAP feature.

Some embodiments may use extrema pulses to guide adaptation of patterns. Some cases may modulate one of the global properties of the stimulation pattern, adjusting all instances of a parameter (e.g., amplitude, frequency or pulse width) by some scalene factor. Some embodiments redefine the pattern itself by keeping the biggest and smallest neural signal (e.g., ECAP) recorded in some range. A pattern may be modulated between a smallest and largest desired value to achieve a range of neural activation, and a certain parameter range, with minimum and maximum values, may be prescribed at programming. Sensing events may be linked to the pattern. In response to a deviation from a desired ECAP recorded (minimum or maximum), other pattern parameters (such as depth, center or period) may be modulated to achieve target levels. The center represents a mean of the parameter. The depth represents the increase or decrease from the mean. The period represents how often the period repeats.

The non-uniform waveform pattern may have a variable pulse parameter with parameter values within a parameter range that includes a minimum parameter value and a maximum parameter value within the parameter range. The sensing window(s) may be automatically assigned to sense a first extrema neural response to the neurostimulation corresponding to the minimum parameter value and a second extrema neural response to the neurostimulation corresponding to the maximum parameter value. The subject matter may further include determining a desired neural response for the automatically assigned sensing window based on the first and second extrema neural responses and using a deviation between the sensed neural response and the desired neural response to control the delivery of the neurostimulation. The delivery of the neurostimulation may be controlled based on the deviation by modulating at least one of a center, depth or period for the variable pulse parameter to provide the desired neural response.

The method may include sweeping neurostimulation through a plurality of parameter values to collect threshold neural response data at one or more threshold parameter values, designating threshold level specifications for the non-uniform waveform pattern based on the one or more threshold parameter values and the collected threshold neural response data, and maintaining the neurostimulation within the threshold level specifications, using the collected threshold neural response data. Sweeping may include sweeping past a perception threshold parameter value, a maximum comfort threshold parameter value, or a discomfort threshold parameter value.

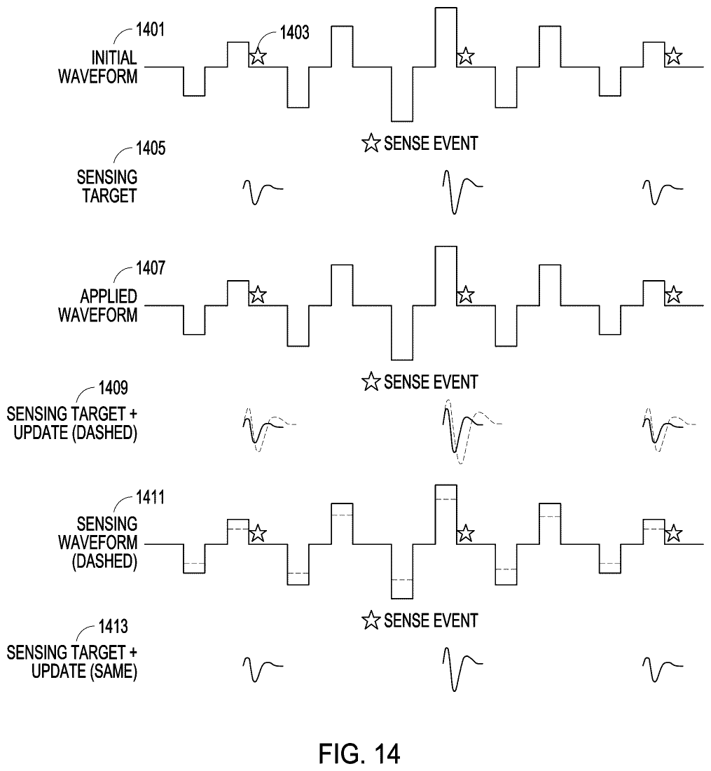

FIG. 14 illustrates, by way of example and not limitation, the use of extrema pulses for determining sense events and modifying the stimulation pattern based on recorded and target sense events. For example, an initial waveform 1401 is illustrated, and the stars 1403 represent sense events at the extrema pulses. Sensing targets 1405, represent targeted values for the sensed signal, for the extrema pulses in the non-uniform waveform are also illustrated. An applied waveform is illustrated at 1407. As illustrated at 1409, a recorded neural signal (dotted line) that is responsive to the applied waveform may be compared to the sensing target (solid line). A modified or updated applied waveform may be applied at 1411, and an updated recorded signal, responsive to the updated applied waveform, may match a sensing target as illustrated at 1413.

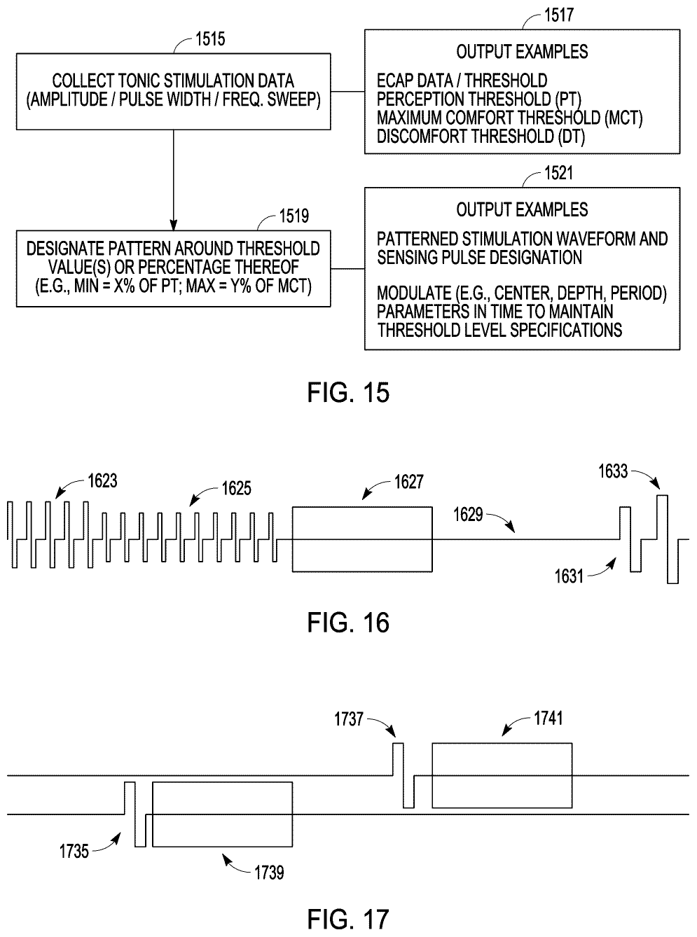

FIG. 15 illustrates, by way of example and not limitation, a clinical workflow using extrema pulses to guide adaptation of patterns. At 1515, tonic stimulation data is collected to capture amplitude, pulse width and a frequency sweep. As generally illustrated at 1517, the workflow may output ECAP data such as an ECAP threshold, a perception threshold (PT), a maximum comfort threshold (MCT), and or a discomfort threshold (DT). At 1519, a pattern may be designated around threshold value(s) or a percentage thereof. For example, the pattern may have a minimum pulse that is x % of the PT or y % of MCT. As generally illustrated at 1521, the workflow may output a pattern stimulation waveform and a sensing pulse designation. The workflow may modulate parameter(s) in time (e.g., center, depth, period) to maintain threshold level specifications. The workflow may be used for subthreshold neurostimulation as well as superthreshold neurostimulation. Some embodiments may use only an ECAP threshold. Other embodiments may use one or more of PT, MCT or DT.

Rather than providing sensing window(s) for a pre-defined non-uniform waveform, some embodiments may design the non-uniform waveform with the sensing pulse(s) (e.g., sensing window(s)). Some embodiments may integrate sensing parameterization with a programmer that is capable of being used to create non-uniform waveform parameters. For example, as discussed earlier, a block-by-block stimulation pattern may be built. A programmer configured for use to build the block-by-block stimulation pattern may be used to insert sense pulses in the non-uniform waveform pattern.

Sensing may be inserted at meaningful epochs in the non-uniform waveform pattern, or at quiescent periods when an evoked potential or a change in local field potentials are expected. Some embodiments may add quiescent period sensing to periods of inactive stim that exceed a certain threshold (e.g., 10 ms or more). Some embodiments may use paired pulse sensing, which may be added by default to when a linkage is created between two programs with stimulation applied at different leads or areas.

FIG. 16 illustrates, by way of example and not limitation, quiescent period sensing in a non-uniform waveform parameter. Different “blocks” may be used to build the waveform, which may include a higher amplitude higher frequency block 1623, a lower amplitude and higher amplitude block 1625. a quiescent period 1627 for the sensing window, a delay 1629, a lower amplitude lower frequency block 1631 and a lower amplitude higher frequency block 1633.

FIG. 17 illustrates, by way of example and not limitation, paired pulse sensing. Pair pulse sensing may show some relationship (positive or negative) for stimulation at one site and at another site. Signals are measured at each site. A second block 1735 may be paired with a first block 1737, and sensing windows 1739, 1741 may be provided for the paired blocks to measure neural signals at each site.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a non-transitory computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
accessing a non-uniform waveform pattern for use to deliver neurostimulation;
automatically assigning, based on the non-uniform waveform pattern, at least one sensing window for the non-uniform waveform pattern;
delivering neurostimulation corresponding the non-uniform waveform pattern;
sensing a neural signal during the at least one sensing window when the neurostimulation is being delivered; and
controlling delivery of the neurostimulation based on the sensed neural signal.

2. The method of claim 1, wherein the non-uniform waveform pattern includes activation pulses that cause a neural response and sub-activation threshold pulses that do not cause the neural response, and the automatically assigning the at least one sensing window includes determining at least one pulse in the non-uniform waveform pattern that corresponds to the neural response and assigning the at least one sensing window to sense the neural response.

3. The method of claim 2, wherein the determining the at least one pulse in the non-uniform waveform pattern includes using a relationship between a neural activation and at least one pulse parameter for the at least one pulse to determine the at least one pulse in the non-uniform waveform pattern that corresponds to the neural response.

4. The method of claim 3, wherein the relationship is between the neural activation and at least one of a pulse amplitude, a pulse frequency or a pulse width.

5. The method of claim 2, wherein the determining the at least one pulse in the non-uniform waveform pattern includes automatically determining a largest pulse amplitude in the non-uniform waveform pattern, and the at least one sensing window is automatically assigned to sense a neural response to the neurostimulation corresponding to the largest pulse amplitude in the non-uniform waveform pattern.

6. The method of claim 2, wherein the determining the at least one pulse in the non-uniform waveform pattern includes automatically selecting multiple pulses in the non-uniform waveform pattern, and the at least one sensing window is assigned to sense a neural response to the neurostimulation corresponding to the automatically selected multiple pulses.

7. The method of claim 6, wherein:
the non-uniform waveform pattern is repeated to provide multiple instances of each pulse in the non-uniform waveform pattern;
the neurostimulation is delivered corresponding the repeated non-uniform waveform pattern; and
the sensing the neural signal includes sensing at least a first neural response to neurostimulation corresponding to a first selected pulse in the repeated non-uniform waveform pattern,
the method further comprising averaging the first neural response to neurostimulation corresponding to at least two instances of the first selected pulse in the repeated non-uniform waveform pattern.

8. The method of claim 7, wherein the averaging the first neural response includes determining a weighted average of the first neural response corresponding to the at least two instances.

9. The method of claim 6, wherein:
the sensing the neural signal includes sensing at least a first neural response to neurostimulation corresponding to a first selected pulse in the non-uniform waveform pattern and a second neural response to neurostimulation corresponding to a second selected pulse in the non-uniform waveform pattern,
the method further comprising determining a weighted average of the first and second neural responses and controlling the delivery of the neurostimulation based on the weighted average.

10. The method of claim 1, wherein the controlling the delivery of the neurostimulation based on the sensed neural signal includes modulating a mean for at least one pulse parameter based on the sensed neural signal.

11. The method of claim 1, wherein:
the non-uniform waveform pattern has a variable pulse parameter with parameter values within a parameter range that includes a minimum parameter value and a maximum parameter value within the parameter range;
the at least one sensing window is automatically assigned to sense a first extrema neural response to the neurostimulation corresponding to the minimum parameter value and a second extrema neural response to the neurostimulation corresponding to the maximum parameter value, and
the method includes:
determining a desired neural response for the automatically assigned at least one sensing window based on the first and second extrema neural responses, and
using a deviation between the sensed neural response and the desired neural response to control the delivery of the neurostimulation.

12. The method of claim 11, wherein the controlling the delivery of the neurostimulation based on the deviation includes modulating at least one of a center, depth or period for the variable pulse parameter to provide the desired neural response.

13. The method of claim 1, further comprising:
sweeping neurostimulation through a plurality of parameter values to collect threshold neural response data at one or more threshold parameter values;
designating threshold level specifications for the non-uniform waveform pattern based on the one or more threshold parameter values and the collected threshold neural response data; and
maintaining the neurostimulation within the threshold level specifications, using the collected threshold neural response data.

14. The method of claim 13, wherein the sweeping the neurostimulation includes sweeping past a perception threshold parameter value, a maximum comfort threshold parameter value, or a discomfort threshold parameter value.

15. The method of claim 1, further comprising identifying a meaningful epoch in the non-uniform waveform, wherein the at least one sensing window is automatically assigned during the identified meaningful epoch.

16. The method of claim 1, further comprising identifying when an evoked potential or a local field potential change is expected, wherein the sensing window is automatically assigned during a quiescent period within the non-uniform waveform pattern when the evoked potential or the change in field potentials is expected.

17. The method of claim 1, further comprising determining a quiescent period within the non-uniform waveform pattern that is longer than a threshold period of time, wherein the sensing window is automatically assigned to the quiescent period that is determined longer than the threshold, wherein the threshold period of time is a user-programmable period of time.

18. The method of claim 1, wherein:
the automatically assigning includes automatically assigning a first sensing window in a first non-uniform waveform pattern and automatically assigning a second sensing window in a second non-uniform waveform pattern;
the delivering neurostimulation includes delivering first neurostimulation corresponding to a first non-uniform waveform pattern at a first stimulation site and delivering second neurostimulation corresponding to a second non-uniform waveform pattern at a second stimulation site;
the sensing the neural signal includes sensing a first neural response to the first neurostimulation within the first sensing window and sensing a second neural response to the second neurostimulation within the second sensing window;
the controlling includes controlling delivery of at least one of the first neurostimulation or the second neurostimulation based on both of the first neural response and the second neural response.

19. A non-transitory machine-readable medium including instructions, which when executed by a machine, cause the machine to perform a method comprising:
accessing a non-uniform waveform pattern for use to deliver neurostimulation;
automatically assigning, based on the non-uniform waveform pattern, at least one sensing window for the non-uniform waveform pattern;
delivering neurostimulation corresponding the non-uniform waveform pattern;
sensing a neural signal during the at least one sensing window when the neurostimulation is being delivered; and
controlling delivery of the neurostimulation based on the sensed neural signal.

20. A system, comprising:
a neural sensor configured to sense a neural signal;
a neurostimulator configured to access a non-uniform waveform pattern and deliver the neurostimulation corresponding to the non-uniform waveform pattern; and
a controller configured to automatically assign, based on the non-uniform waveform pattern, at least one sensing window for the non-uniform waveform pattern, control the neural sensor to sense the neural signal during the at least one sensing window when the neurostimulation is delivered, and control delivery of the neurostimulation from the neurostimulator based on the sensed neural signal.
